(19) United States
(12) Patent Application Publication
(10) Pub. No.: US 2023/0135374 A1
(43) Pub. Date: May 4, 2023

(54) GRAPHENE-FUNCTIONALIZED SENSOR SURFACES AND RELATED METHODS

(71) Applicant: GRAPHENE DX, INC., Boston, MA (US)

(72) Inventors: Emily Borrello, Somerville, MA (US); Tom Ruby, Boston, MA (US); Thomas Feltrup, South Weymouth, MA (US)

(21) Appl. No.: 17/969,614

(22) Filed: Oct. 19, 2022

Related U.S. Application Data
(60) Provisional application No. 63/273,853, filed on Oct. 29, 2021.

(51) Int. Cl. G01N 33/487

(52) U.S. Cl. CPC G01N 33/48714; B82Y 15/00

(57) ABSTRACT

Systems and methods of functionalizing a graphene sensor surface are provided. A representative method of functionalizing the graphene sensor surface includes providing a graphene layer including a sensor surface. The method may include binding a plurality of molecular complexes to the sensor surface of the graphene layer using a buffer solution. Each molecular complex may include a linker molecule configured to couple to the sensor surface at a first linker position, and a binding molecule coupled to the linker molecule at a second linker position different from the first linker position. The representative method further includes coupling one or more detector molecules to a first subset of the molecular complexes and coupling one or more passivation agents to a second subset of the molecular complexes. At least some of the molecular complexes of the first subset are different from those of the second subset.

### CROSS-REFERENCE TO RELATED APPLICATIONS

This application claims priority to U.S. Prov. App. No. 63/273,853, filed on Oct. 29, 2021, and entitled “Graphene-Functionalized Sensor Surfaces And Related Methods,” which is incorporated herein by reference.

### TECHNICAL FIELD

The present disclosure relates generally to functionalizing a graphene-based sensor for analyzing biological test samples.

### BACKGROUND

Current methods of functionalizing the surface of a graphene-based sensor pose challenges for scalable manufacturing. In particular, a standard approach to functionalizing the sensor surface includes a stepwise process in which each molecule is added to the sensor surface independently. This approach can result in binding molecules, such as antibodies, having random orientations on the sensor's surface. Random orientations affecting the molecules responsible for the basic sensing function—i.e., binding to chemical species to be detected—impairs overall sensing performance. Further, existing methods of sensor assembly and functionalization can degrade the sensor surface, further compromising overall sensor accuracy.

Accordingly, there is a need for improved approaches to functionalizing a sensor surface.

### SUMMARY

The present disclosure is directed to methods of functionalizing the surface of a graphene-based sensor, as well as to the finished sensors themselves. In particular, the present disclosure describes techniques for conveniently binding molecular complexes to a graphene sensor surface. The molecular complexes control the orientation of one or more detector molecules, thereby improving the performance of the graphene-based sensor (e.g., improving the ability of a detector molecule to recognize and bind to a target biomarker). Additionally, approaches to functionalizing a sensor surface described herein reduce degradation of the graphene sensor surface by using a water-based solution (e.g., a phosphate-buffered solution) to bind the molecular complexes to a graphene sensor surface. By reducing the degradation of the graphene sensor surface, embodiments of the present invention maintain the electrical properties of the graphene-based sensor, thereby improving overall performance.

In one aspect, a method of functionalizing a graphene sensor surface is provided. The method includes, in various embodiments, providing a graphene layer including a surface and binding a plurality of molecular complexes to the surface of the graphene layer. In some embodiments, the plurality of molecular complexes are bonded to the surface using a buffer solution. Each molecular complex includes a linker molecule configured to couple to the sensor surface at a first linker position, and a binding molecule coupled to the linker molecule at a second linker position different from the first linker position. Embodiments of the method further include coupling one or more detector molecules to a first subset of the molecular complexes and coupling one or more passivation agents to a second subset of the molecular complexes. At least some of the molecular complexes of the first subset are, in various embodiments, different from molecular complexes of the second subset.

In some embodiments, the detector molecule(s) coupled to the first subset of the molecular complexes are each coupled to a binding molecule of the associated molecular complex. In some embodiments, the one or more passivation agents coupled to the second subset of the molecular complexes are each coupled to a binding molecule of the associated molecular complex.

In some embodiments, the method includes coupling the molecular complex to the sensor surface before coupling one or more detector molecules to the first subset of the molecular complexes. Alternatively, in some embodiments, the method includes coupling the molecular complex to the sensor surface after coupling one or more detector molecules to the first subset of molecular complexes. In some embodiments, the method further includes coupling the molecular complex to the sensor surface before coupling one or more passivation agents to the second subset of molecular complexes. Alternatively, in some embodiments, the method includes coupling the molecular complex to the sensor surface after coupling one or more passivation agents to the second subset of molecular complexes.

In some embodiments, binding the plurality of molecular complexes to the sensor surface of the graphene layer includes generating a molecular complex mixture by dissolving, at least in part, the molecular complexes in a buffer solution, disposing the molecular complex mixture on the sensor surface of the graphene layer, and incubating the mixture and the graphene layer for a first predetermined amount of time at a predetermined temperature. In some embodiments, the first predetermined amount of time is at least 14 hours and the predetermined temperature is 4° C. In some embodiments, the molecular complex mixture has a molar concentration of approximately 5 millimolar.

In some embodiments, coupling the detector molecule(s) to the first subset of molecular complexes includes generating a detector molecule mixture by dissolving, at least in part, the one or more detector molecules in a buffer solution, disposing the detector molecule mixture on the first subset of molecular complexes, and incubating the detector molecule mixture and the first subset of molecular complexes for a second predetermined amount of time at a predetermined temperature. In some embodiments, the detector molecule mixture has a predetermined mass concentration of 10 μg/ml.

In some embodiments, coupling the one or more passivation agents to the second subset of molecular complexes includes generating a passivation agent mixture by dissolving, at least in part, the passivation agent(s) in the buffer solution, disposing the passivation agent mixture on the second subset of molecular complexes, and incubating the passivation agent mixture and the second subset of molecular complexes for a second predetermined amount of time at a predetermined temperature. In some embodiments, the passivation agent mixture has a predetermined concentration of 3% and a pH of 8.

In some embodiments, coupling the detector molecule(s) to the first subset of molecular complexes includes generating one or more modified detector molecules by adding a tag to each detector molecule and coupling the thus-modified detector molecule(s) to one or more binding molecules of the first subset of molecular complexes via the tag(s).

In some embodiments, coupling the detector molecule(s) to the first subset of molecular complexes includes, while generating an electric field, coupling the detector molecule(s) to one or more binding molecules of the first subset of the molecular complexes.

In some embodiments, the first subset molecular complexes and the second subset of molecular complexes have a predetermined ratio. In some embodiments, the plurality of molecular complexes is configured to control the orientation of the detector molecule(s). In some embodiments, the detector molecules antibodies, enzymes, or other proteins. In some embodiments, the binding molecule includes or consist of protein A, protein G, and/or protein L. In some embodiments, the buffer solution is phosphate-buffered saline. In some embodiments, the passivation agent(s) are amino-PEG5-alcohol (APA or PEG5). In some embodiments, the linker molecule is 1-pyrenebutanoic acid succinimidyl ester (PBASE).

In another aspect, a graphene-based sensor is provided. The graphene-based sensor includes a graphene sensor surface that has been functionalized by binding a plurality of pre-conjugated molecular complexes thereto. The pre-conjugated molecular complexes may be coupled to the graphene layer using a using a buffer solution. In some embodiments, a first subset of the pre-conjugated molecular complexes includes one or more linker molecules, one or more binding molecules, and one or more detector molecules configured to detect a target biomarker. The linker molecule may be configured to couple to the sensor surface at a first linker position. The binding molecule may be configured to couple to the detector molecule, and also couple to the linker molecule at a second linker position different from the first linker position. In some embodiments, a second subset of the pre-conjugated molecular complexes includes one or more linker molecules, one or more binding molecules, and one or more passivation agents configured to block or hinder non-specific molecules from the graphene sensor surface. The linker molecule may be configured to couple to the sensor surface at a first linker position. The binding molecule may be configured to couple to the passivation agent, and couple to the linker molecule at a second linker position different from the first linker position. At least some of the molecular complexes of the first subset may be different from molecular complexes of a second subset.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims.

### BRIEF DESCRIPTION OF THE DRAWINGS

So that the present disclosure can be understood in greater detail, a more particular description may be had by reference to the features of various embodiments, some of which are illustrated in the appended drawings. The appended drawings, however, merely illustrate pertinent features of the present disclosure and are therefore not to be considered limiting, for the description may admit to other effective features as the person of skill in this art will appreciate upon reading this disclosure.

FIG. 1 illustrates a graphene-based sensor functionalized using one or more molecular complexes, in accordance with some embodiments.

FIGS. 2A-2E illustrate a standard process for functionalizing a graphene-based sensor.

FIGS. 3A-3D illustrate pre-conjugation of one or more molecular complexes, in accordance with some embodiments.

FIGS. 4A-4D illustrate a method of functionalizing a sensor surface of a graphene-based sensor using one or more molecular complexes, in accordance with some embodiments.

FIGS. 5A-5B illustrate a single-step method of functionalizing a sensor surface of a graphene-based sensor using one or more molecular complexes, in accordance with some embodiments.

FIG. 6 is a flow diagrams illustrating a method of functionalizing a sensor surface, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote like features throughout the specification and figures.

### DETAILED DESCRIPTION

Numerous details are described herein in order to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not been described in exhaustive detail so as to avoid obscuring pertinent aspects of the embodiments described herein.

The present teachings are generally directed to a graphene-based sensor that can be functionalized with a plurality of antibodies for detecting and/or analyzing one or more biomarkers. The term “biomarker” as used herein refers to a molecular species with a biological function. As discussed in more detail below, a sensor surface of the graphene-based sensor can be functionalized utilizing pre-conjugated molecules that are added and coupled to the sensor surface. The pre-conjugated molecules are configured to couple to one or more detector molecules (e.g., antibodies, enzymes, other proteins, etc.) that enable the graphene-based sensor to recognize and bind to specific target biomarkers for analysis. In particular, use of the pre-conjugated molecules improves the performance of the graphene-based sensor by controlling the orientation of the detector molecules coupled to the sensor surface, improving the detector molecules' ability to recognize and bind to specific target biomarkers. Further, the pre-conjugated molecules can be coupled to the sensor surface of the graphene-based sensor without degrading that surface.

The graphene-based sensor described herein is configured to receive a test sample and detect whether a target biomarker is present therein. For example, if the target biomarker is present in the test sample under investigation, the interaction of the biomarker with the graphene-based sensor may cause a change in at least one electrical property of the underlying graphene layer, e.g., its DC electrical resistance. Such a change in the electrical property of the graphene layer can be measured and used to quantify the amount of the biomarker in the sample.

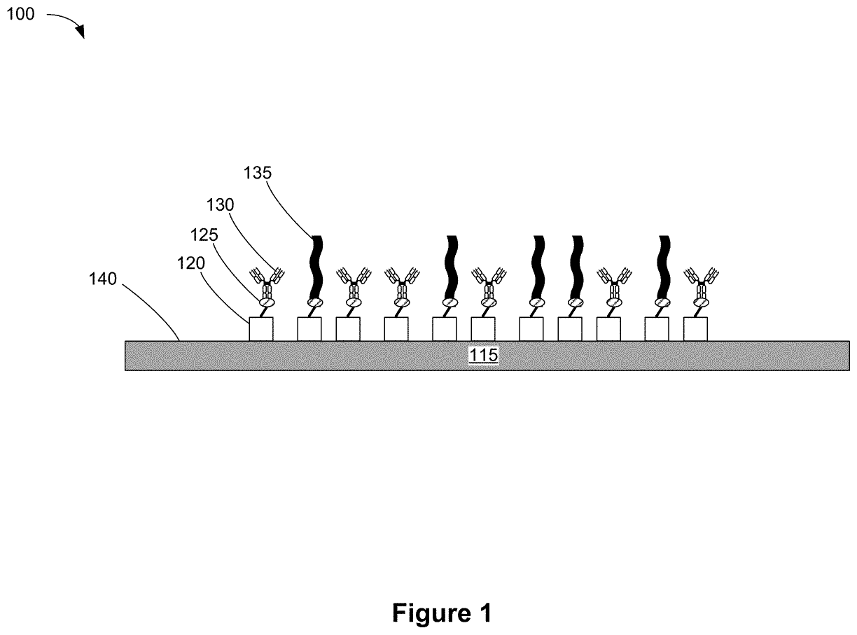

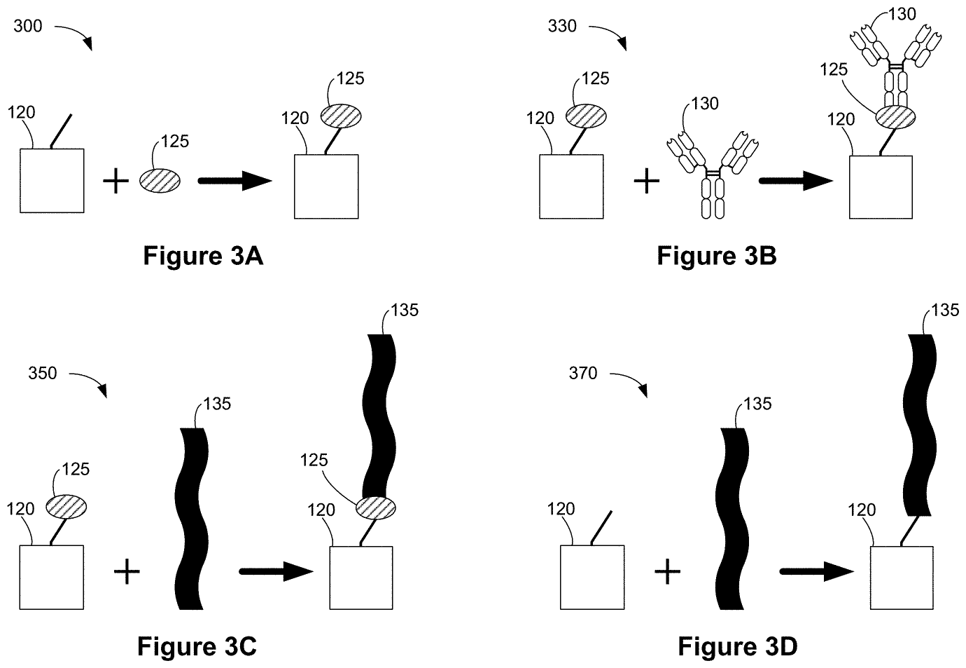

FIG. 1 illustrates a graphene-based sensor 100 functionalized using one or more molecular complexes, in accordance with some embodiments. As described above, the graphene-based sensor 100 is configured to analyze a test sample. In some embodiments, the test sample is a urine sample, a blood sample, a saliva sample, and/or any other biological substance (typically in liquid or gaseous form). In some embodiments, the graphene-based sensor 100 analyzes the test sample to detect and/or quantify one or more target biomarkers, bacteria, allergens, pathogens, proteins, glutens, toxins, etc. In some embodiments, the graphene-based sensor 100 includes a graphene layer 115 and one or more molecular complexes. Each molecular complex can be a combination of two or more linker molecules 120, one or more binding molecules 125, one or more detector molecules 130, and/or one or more passivating agents 135. In some embodiments, one or more molecular complexes are pre-conjugated as described below in reference to FIGS. 3A-3D.

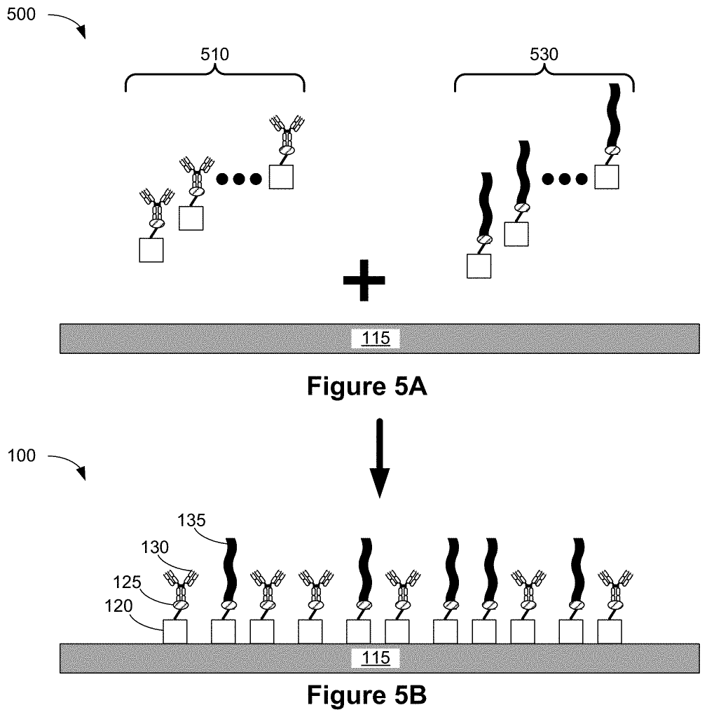

In some embodiments, the graphene layer 115 is functionalized with one or more detector molecules 130 via the one or more molecular complexes. In particular, in some embodiments, one or more molecular complexes are coupled to the graphene layer 115 and the one or more detector molecules 130 are coupled to the one or more molecular complexes. The functionalization process described below with reference to FIGS. 3A-5B allows for control of the orientation of the one or more detector molecules 130 (when coupled to the one or more molecular complexes). By controlling the orientation of the detector molecule(s) 130, the functionalization process described herein improves the performance of the graphene-based sensor 100 by enabling the detector molecules 130 to recognize and bind to a target biomarker efficiently and effectively. Further, the functionalization process described herein reduces or eliminates degradation of the graphene layer 115 sensor surface 140 when the one or more molecular complexes are coupled to the sensor surface 140. By reducing the degradation of the graphene layer 115, the electrical properties of the graphene layer 115 are improved to maintain more consistent and reliable performance of the sensor 100. Additionally, the functionalization process described herein enables the manufacture of graphene-based sensor 100 to be reduced to a one-step functionalization of the surface (as described below in reference to FIGS. 5A-5B), which makes at-scale manufacturing of the graphene-based sensor 100 feasible and cost-effective.

In some embodiments, a linker molecule 120 is a covalent and/or non-covalent chemical linker, i.e., bonds covalently and/or non-covalently (e.g., ionically or via hydrogen bonding) to the sensor surface and to a detector molecule; that is, the mode of attachment may include, one or more covalent bonds, one or more non-covalent bonds, or a combination. In some embodiments, the linker molecule is a pyrene linker, such as 1-pyrenebutanoic acid succinimidyl ester (PBASE) or any other pyrene derivative that behaves as a heterobifunctional linker. In some embodiments, the linker molecule 120 is configured to couple to the sensor surface 140 at a first linker position. The linker molecule 120 may also couple to a binding molecule 125 at a second linker position different from the first linker position. In some embodiments, the binding molecule 125 is a fragment crystallizable (Fc) binding peptide or aptamer. For example, the binding molecule 125 may be or include protein A, protein G, protein L, and/or other Fc-binding peptide or aptamer.

In some embodiments, the binding molecule 125 is capable of coupling (covalently or non-covalently) not only to a detector molecule 130 (e.g., anti-troponin antibodies) but alternatively to a passivation agent 135; accordingly, the second linker position may alternatively bind a detector molecule 130 or the passivation agent 135 as shown in FIG. 1. In some embodiments, the detector molecule(s) 130 include or consist of an antibody, enzyme, other protein, or other chemical species capable of specifically recognizing and binding (covalently or non-covalently) a target biomarker. In some embodiments, the passivation agent 135 is APA (PEG5), Tween, BLOTTO, bovine serum albumin (BSA), and/or gelatin. The passivation agent can inhibit, and preferably prevent, the interaction of a sample of interest introduced onto the graphene layer 115 with areas of the graphene layer 115 that are not functionalized with the one or more detector molecules 130. This can in turn lower the noise in electrical signals generated as a result of the interaction of the analyte of interest with the detector molecules 130.

By way of example, in this embodiment, the graphene layer 115 is functionalized with one or more detector molecules 130 that exhibit specific binding to any of troponin (e.g., a particular isoform of troponin), C-reactive protein, B-type natriuretic peptide, or myeloperoxidase. In some embodiments, the detector molecule(s) 130 (e.g., anti-biomarker antibodies) are monoclonal antibodies that exhibit specific binding to a particular isoform of the biomarker, e.g., a specific isoform of troponin. In other embodiments, the one or more detector molecule 130 (e.g., anti-biomarker antibodies) can be polyclonal antibodies that exhibit binding to multiple isoforms of the biomarker. By way of example, in some embodiments, the graphene layer 115 can be functionalized with cardiac troponin T (cTnT) and/or cardiac troponin I (cTnI).

In some embodiments, the one or more molecular complexes cover a fraction of, or the entire, surface of the graphene layer 115. In some embodiments, the fraction can be at least about 60%, at least about 70%, at least about 80%, or 100% of the surface of the graphene layer 115. The remainder of the surface of the graphene layer 115 (i.e., the surface areas not functionalized with the detector molecule 130) can be passivated via one or more passivation agents 135. Additionally, or alternatively, in some embodiments, the non-functionalized graphene areas can be passivated via a passivation layer. By way of example, passivation of the non-functionalized portions of the graphene layer 115 can be achieved, e.g., via incubation with 0.1% Tween. As described above, passivation can lower the noise in the electrical signals generated as a result of the interaction between the analyte of interest and the detector molecules 130.

In some embodiments, the graphene-based sensor 100 is disposed on (e.g., bonded to) an underlying substrate. The underlying substrate can be formed of a variety of different materials, such as, silicon, plastic, metal, polymeric materials (such as polyurethane or polyethylene terephthalate), or glass, among others. In some embodiments, the graphene layer 115 is disposed over an underlying silicon oxide (SiO₂) layer, which can in turn be formed as a thin layer of or on a silicon substrate (e.g., a layer having a thickness in a range of a 200 nm to about 10 μm). In some embodiments, the graphene layer 115 can be deposited on an underlying silicon substrate using any of a variety of techniques known in the art. By way of example, chemical vapor deposition (CVD) can be employed to deposit the graphene layer 115 on an underlying copper substrate. The graphene-coated copper substrate can then be disposed on (e.g., bonded to) a silicon oxide layer of a silicon wafer, and the copper can be removed via chemical etching. In some embodiments, the graphene layer 115 is deposited on the underlying substrate as an atomic monolayer, while in other embodiments the graphene layer 115 includes multiple atomic layers.

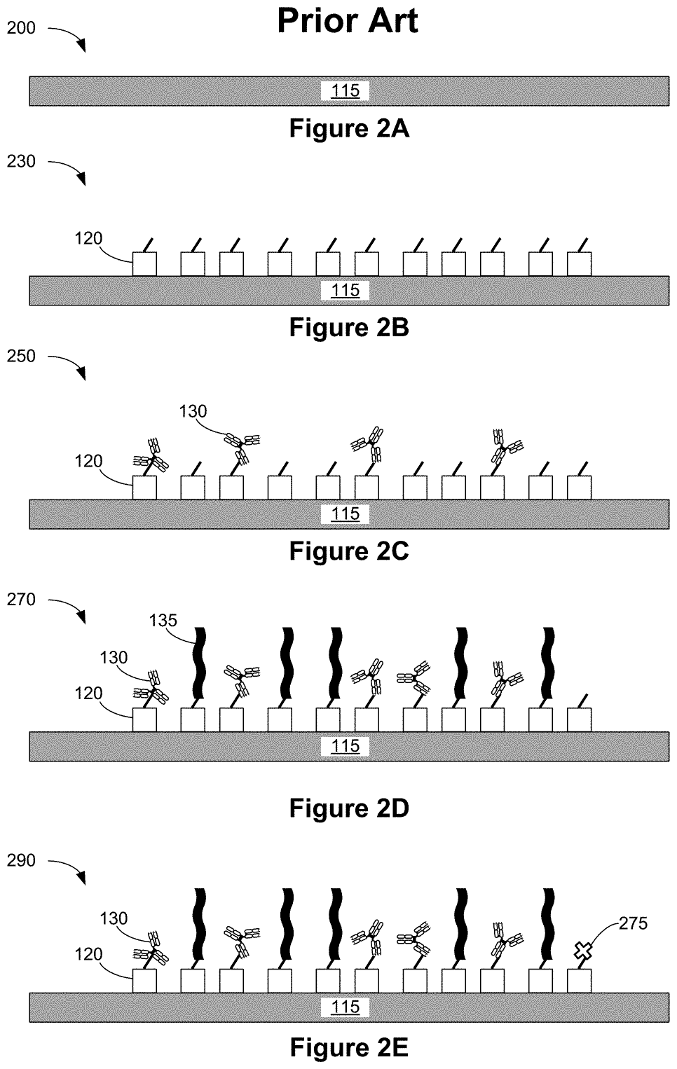

FIGS. 2A-2E illustrate a standard process for functionalizing a graphene-based sensor. The standard process for functionalizing the graphene-based sensor includes, in a first operation 200, providing a graphene layer 115. The standard process further includes, in a second operation 230, disposing one or more linker molecules 120 on the graphene layer 115. The graphene layer 115 is incubated with the linker molecule(s) 120 (e.g., a 5 nM solution of PBASE in a dimethyl sulfoxide (DMSO) solution (or a dimethylformamide (DMF) solution, or methanol) for a first time period (e.g., an hour) at a predetermined temperature (e.g., room temperature), causing the linker molecule(s) to bind to the graphene surface.

In a third operation 250, the linker-modified graphene layer is then incubated with the detector molecule(s) 130 of interest in, e.g., a sodium carbonate bicarbonate solution (e.g., at a pH of 9.5 and with a mass concentration of at least 60 μg/ml) for a second time period (e.g., two hours) at the predetermined temperature. In the third operation 250, the detector molecule(s) 130 bind to the linker molecules 120 of the linker-modified graphene layer. The detector molecule(s) 130 are not uniformly coupled to the linker molecules 120. In particular, the orientations of the detector molecules 130 are not the same across different linker molecules 120 and, in some cases, the detector molecules 130 are coupled to linker molecules 120 at inefficient orientations. The inconsistencies in the orientations of the detector molecules 130 lower the performance of a graphene-based sensor functionalized using the illustrated standard process.

In a fourth operation 270, the linker-modified graphene layer coupled to one or more detector molecules 130 is further incubated with a passivation agent 135. Under the standard process, the passivation agent is incubated in a phosphate-buffered solution (PBS) for a third predetermined time period (e.g., 30 minutes) at the predetermined temperature. The PBS can have a pH of, e.g., 11. Further, a fifth operation 290 of the standard process includes rinsing the linker-modified graphene layer coupled to one or more detector molecules 130 and passivation agents 135 with deionized (DI) water and PBS. In order to quench the unreacted succinimidyl ester groups, the modified graphene layer can be incubated with ethanolamine (e.g., 0.1 M solution at a pH of 11 for another predetermined time period (e.g., 30 minutes) at the predetermined temperature).

The performance of graphene-based sensors functionalized using the illustrated standard process can vary. In particular, the inability to control the orientation of the detector molecules 130 can result in poor sensor performance (e.g., providing inconsistent readings, generating false positives, and/or failing to detect a target biomarker). Further, the different operations of the standard process can degrade the graphene layer 115 and change its electrical properties, resulting in inaccurate readings or detection of spurious electrical signals.

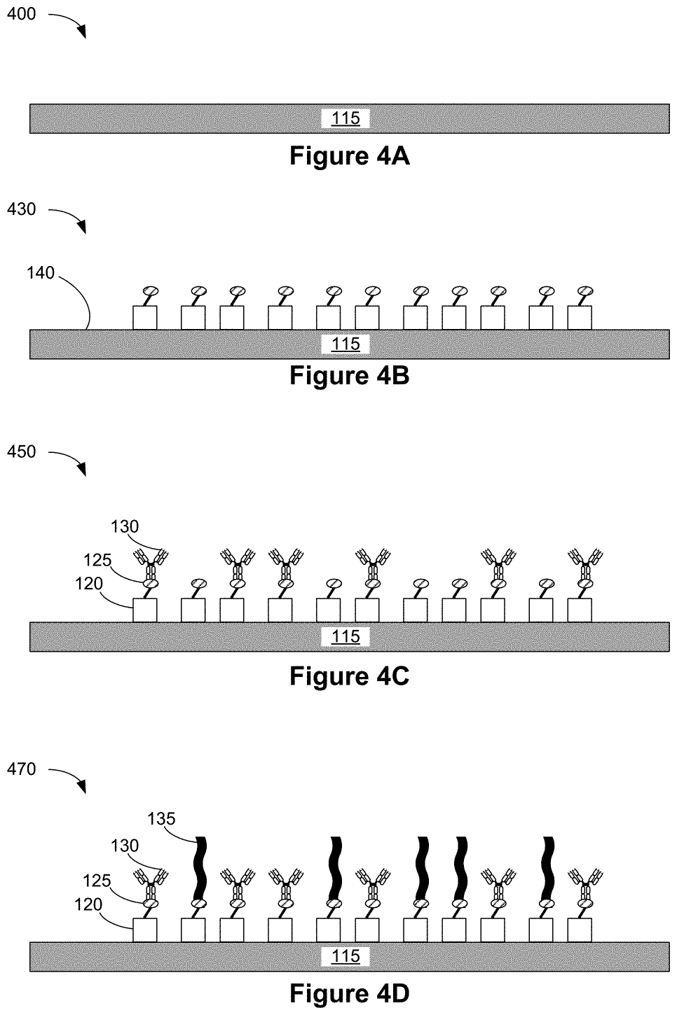

FIGS. 3A-3D illustrate pre-conjugation of one or more molecular complexes in accordance with some embodiments. In particular, one or more molecules (e.g., a linker molecule 120, a binding molecule 125, a detector molecule 130, and/or a passivation agent 135) are conjugated into new molecular complexes before coupling to a sensor surface 140 of the graphene-based sensor 100 (FIG. 1). The coupling of molecular complexes to the sensor surface 140 of the graphene-based sensor 100 is discussed in detail below in reference to FIGS. 4A-5B.

FIG. 3A illustrates a first molecular complex 300 in accordance with some embodiments, e.g., a linker molecule 120 conjugated with a binding molecule 125. The first molecular complex 300 forms a duplex conjugate in which the binding molecule 125 binds to the linker module 120 at one linker position, and the linker module binds to the sensor surface 140 of the graphene-based sensor 100 at another (e.g., opposed) linker position.

FIG. 3B illustrates conjugation of the binding molecule 125 with a detector molecule 130 to form a modified molecular complex 330 in which binding molecule 125 binds to the linker module 120 at one linker position and further binds to the detector molecule 130 at a different linker position. As described above with reference to FIG. 1, in some embodiments, the binding molecule 125 is an Fc binding peptide or aptamer. The Fc binding peptide or aptamer is configured to bind to an Fc region of the detector molecule 130, which enables the detector molecule 130 to be oriented on the sensor surface 140 to improve the performance of the graphene-based sensor 100. In this configuration, the linker module 120 couples the sensor surface 140 of the graphene-based sensor 100 via the first linker position opposite the second linker position. In some embodiments, the modified complex 330 is conjugated before it is coupled to the sensor surface 140. Alternatively, in some embodiments, the modified complex 330 is formed while the molecular complex 300 is coupled to the sensor surface 140. In other words, a detector molecule 130 can be coupled to the molecular complex 300 before or after the complex 300 has been coupled to the sensor surface 140.

As shown in FIG. 3C, an alternative molecular complex 350 may be formed by conjugation of the molecular complex 300 with a passivation agent 135. The binding molecule 125 binds to the linker module 120 and further binds to the passivation agent 135. In this configuration, the linker module 120 is configured to couple the sensor surface 140 of the graphene-based sensor 100 via a linker position different from (e.g., opposed to) where the passivation agent 135 is bound. In some embodiments, the molecular complex 350 is formed before it is coupled to the sensor surface 140. Alternatively, in some embodiments, the complex 350 is formed after the complex 300 has been coupled to the sensor surface 140. In other words, a passivation agent 135 can be coupled to the first molecular complex 300 before or after the molecular complex 300 has been coupled to the sensor surface 140.

FIG. 3D illustrates an alternative molecular complex 370 in which the passivation agent 135 directly coupled to the linker molecule 120 (rather than via the binding molecule 125). The linker module 120 is still configured to couple the sensor surface 140 of the graphene-based sensor 100 via the first linker position.

Although not shown, another alternative molecular complex includes a detector molecule 130 directly coupled to the linker molecule 120 (rather than via the binding molecule 125). The linker module 120 is still configured to couple the sensor surface 140 of the graphene-based sensor 100 via the first linker position.

FIGS. 4A-4D illustrate a method of functionalizing the sensor surface of a graphene-based sensor using one or more molecular complexes. A graphene layer 115 is provided (operation 400) and, in an operation 430, a plurality of molecular complexes as described above is exposed to the surface 140 of the graphene layer 115 in a buffer solution (e.g., PBS). Each molecular complex includes a linker molecule 120 configured to couple to the sensor surface 140 at a first linker position and a binding molecule 125 coupled to the linker molecule 120 at a second linker position different from the first linker position. In some embodiments, the molecular complexes coupled to the sensor surface 140 of the graphene layer 115 have the form of the molecular complex 300. In some embodiments, the molecular complexes are coupled to the sensor surface 140 before coupling one or more detector molecules 130 to some or all of the plurality molecular complexes. Alternatively, the binding molecule 125 (e.g., an antibody-binding molecule) may be bound to the sensor surface 140 directly or through a linker molecule 120. The linker molecule 120 can be pre-conjugated to the binding molecule 125 before it is coupled to the sensor surface 140.

In some embodiments, coupling (e.g., binding) the molecular complexes to the sensor surface 140 of the graphene layer 115 includes generating a molecular complex mixture by dissolving, at least in part, the molecular complexes in a buffer solution, which is applied to the sensor surface 140 of the graphene layer 115. The mixture is incubated for a first predetermined amount of time at a predetermined temperature, causing the molecular complexes to bind to the sensor surface 140. In some embodiments, the first predetermined amount of time is at least 14 hours and the predetermined temperature is 4° C. Alternatively, the first predetermined amount of time may be overnight. In some embodiments, the molecular complex mixture has a molar concentration of approximately 5 millimolar.

In some embodiments, in operation 450, detector molecules 130 are coupled to a first subset of the graphene-bound molecular complexes via a binding molecule 125 of the molecular complexes. In some embodiments, this is accomplished by dissolving, at least in part, the detector molecules in a buffer solution (e.g., PBS), applying the mixture to the bound molecular complexes, and incubating for a second predetermined amount of time at a predetermined temperature. In some embodiments, the detector molecule mixture has a mass concentration of 10 μg/ml. The molecular complexes may be selected and bound to the graphene surface so as to control the orientation of the detector molecule(s) 130 when they bind (e.g., such that the detector molecules 130 couple to the binding molecule 125 at the Fc region). This may be accomplished, e.g., using antibody-binding molecules to control the orientation of the antibodies or other detector molecules. In this way, the detector molecules 130 recognize and bind to specific target biomarkers in an optimal orientation (e.g., perpendicular to the sensor surface 140).

Alternatively, coupling the detector molecule(s) 130 to the first subset of molecular complexes may include generating one or more modified detector molecules by adding a tag (not shown) to the detector molecule(s) 140, and coupling the tagged detector molecules to binding molecules 125 of the first subset of molecular complexes. For example, antibody binding can be indirect through the addition of a tag in the antibody sequence, with the tag binding to the binding molecule 125. In some embodiments, the tag can be bound via another molecule directly to the sensor surface 140. An example of such a system includes a biotin-streptavidin system. Antibodies or other detector proteins can be biotinylated at specific sites of the molecule sequence. These modified detector molecules are then added to a streptavidin-coated sensor surface.

Alternatively, in some embodiments, coupling the detector molecule(s) 130 to the first subset of molecular complexes includes, while generating an electric field, coupling the detector molecule(s) to one or more binding molecules of the first subset of molecular complexes. The electric field can be used to orient the antibody or other detector molecule on the sensor surface before or during binding.

In some embodiments, at operation 470, one or more passivation agents 135 are coupled to a second subset of the plurality of molecular complexes. The first and second subsets of molecular complexes may be completely distinct or may overlap, in the latter case with some of the molecular complexes binding to both a detector molecule and a passivation agent. In some embodiments, the passivation agents 135 are coupled to molecular complexes via the associated binding molecule 125 (see FIG. 3C). In some embodiments, the molecular complexes are coupled to the sensor surface 140 before the passivation agents 135 are coupled to the second subset thereof.

In some embodiments, coupling the passivation agents 135 to the second subset of molecular complexes includes generating a passivation agent mixture by dissolving, at least in part, the passivation agents in a buffer solution (e.g., PBS), applying the mixture to the molecular complexes, and incubating for a second predetermined amount of time at a predetermined temperature. In some embodiments, the passivation agent mixture has a concentration of 3% and a pH of 8. In some embodiments, the first subset of the plurality of molecular complexes and the second subset of the plurality of molecular complexes have a predetermined ratio. For example, the first subset of molecular complexes (coupled to the detector molecule(s) 130) can cover 30%, 60%, 80%, or 100% of the graphene surface and the second subset (coupled to the one or more passivation agents 135) can cover 70%, 40%, 20%, or 0% of the graphene surface, respectively. The molecular complexes binding passivation agents may be bound to the graphene surface in regions distinct from the regions where molecular complexes binding detector molecules are bound, or they may be interspersed.

FIGS. 5A and 5B illustrate a single-step method of functionalizing a sensor surface of a graphene-based sensor using molecular complexes that have been pre-conjugated. In this way, the process for functionalizing the sensor surface 140 of a graphene-based sensor 100 is streamlined.

In some embodiments, molecular complexes are coupled to the sensor surface 140 after detector molecules 130 have been coupled to a first subset of the molecular complexes (FIGS. 4A-4D). For example, one or more molecular complexes 510 (e.g., having the form of the molecular complex 330) can be prepared prior to being coupled to the sensor surface 140. Similarly, in some embodiments, the molecular complexes are coupled to the sensor surface 140 after one or more passivation agents 135 (FIGS. 4A-4D) have been coupled to a second subset thereof. For example, one or more molecular complexes 530 (e.g., having the form of the molecular complex 350) can be prepared prior to being coupled to the sensor surface 140.

In operation 500, a mixture of molecular complexes 510, 530 is dissolved in a water-based solution, such as PBS, which is then applied to the sensor surface 140. The mixture is incubated over the surface for a predefined period of time at a suitable temperature (e.g., at least 15 minutes at a temperature of 4° C.). This allows the sensor surface 140 of the graphene-based sensor 100 to be functionalized in a single step.

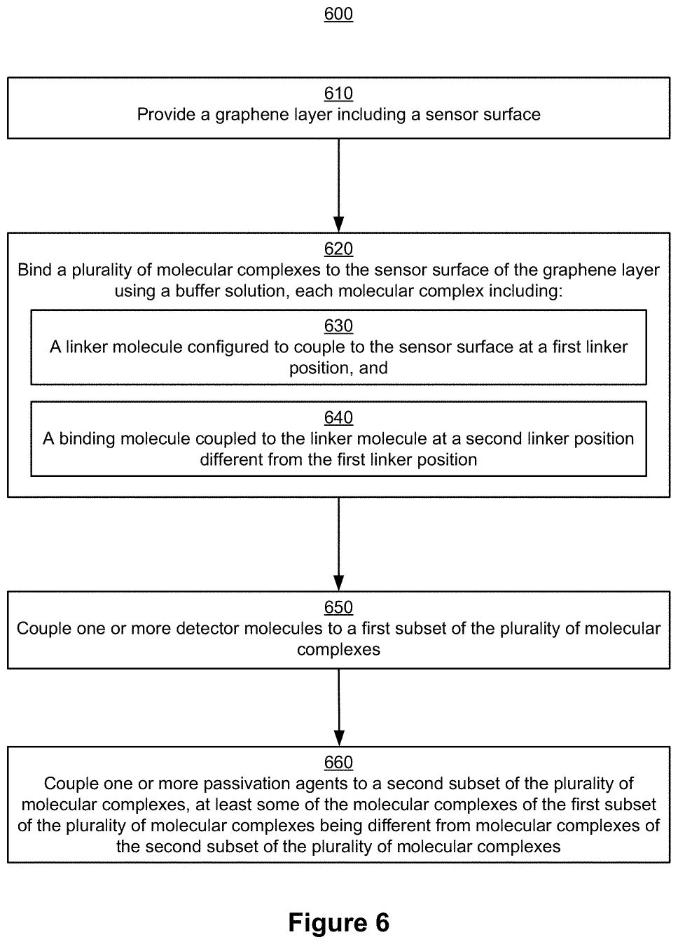

FIG. 6 illustrates a method 600 of functionalizing a sensor surface. Operations (e.g., steps) of the method 600 may be performed to functionalize a graphene sensor surface 100 as described above with reference to FIGS. 3 and 4. The foregoing method descriptions and the process flow diagrams are provided merely as illustrative examples and are not intended to require or imply that the operations of the various embodiments must be performed in the order presented. As will be appreciated by one of skill in the art the steps in the foregoing embodiments may be performed in any order. Words such as “then,” “next,” etc. are not intended to limit the order of the steps; these words are simply used to guide the reader through the description of the methods. Although process flow diagrams may describe the operations as a sequential process, many of the operations can be performed in parallel or concurrently. In addition, the order of the operations may be re-arranged. A process may correspond to a method, a function, a procedure, a subroutine, a subprogram, etc. When a process corresponds to a function, its termination may correspond to a return of the function to the calling function or the main function.

The method 600 includes providing (step 610) a graphene layer including a sensor surface. The method 600 further includes binding (step 620) molecular complexes to the sensor surface of the graphene layer using a buffer solution. Each molecular complex includes a linker molecule configured to couple (step 630) to the sensor surface at a first linker position, and a binding molecule coupled (step 640) to the linker molecule at a second linker position different from the first linker position. For example, as shown and described above in reference to FIG. 3A, in some embodiments, a molecular complex includes at least a linker molecule 120 and a binding molecule 125. In some embodiments, the linker molecule is a pyrene linker, such as PBASE or any other pyrene derivative that operates as a heterobifunctional linker. In some embodiments, the binding molecule 125 is an Fc binding peptide or aptamer. In some embodiments, the binding molecule 125 is one of protein A, protein G, or protein L.

Binding the plurality of molecular complexes to the sensor surface of the graphene layer may include generating a molecular complex mixture by dissolving, at least in part, the molecular complexes in a buffer solution, applying the mixture to the sensor surface of the graphene layer, and incubating for a first predetermined amount of time at a predetermined temperature. In some embodiments, the first predetermined amount of time is overnight, and the predetermined temperature is 4° C. In some embodiments, overnight means at between 12-20 hours. In some embodiments, the molecular complex mixture has a molar concentration of approximately 5 millimolar. In some embodiments, the buffer solution is phosphate-buffered saline.

The method 600 includes coupling (step 650) detector molecules to a first subset of the plurality of molecular complexes. In some embodiments, the detector molecule includes or consists of an antibody, an enzyme, another type of protein, or other molecule capable of specifically recognizing and binding a target biomarker. In some embodiments, the molecular complexes control the orientation of the detector molecule when coupled thereto. For example, as shown and described above with reference to FIGS. 1 and 3A-5B, the molecular complexes control the orientation of the detector molecules 130 such that the Fc region of the detector molecules 130 couples to the plurality of molecular complexes. By controlling the orientation of the detector molecules 130, the method 600 improves the overall performance of the sensor.

In some embodiments, coupling the detector molecules to the first subset of molecular complexes includes generating a by dissolving, at least in part, the detector molecules in a buffer solution, applying the mixture to the first subset of molecular complexes, and incubating for a second predetermined amount of time at a predetermined temperature. In some embodiments, the second predetermined amount of time is at least 15 minutes, and the predetermined temperature is 4° C. In some embodiments, the detector molecule mixture has a predetermined mass concentration of 10 μg/ml. In some embodiments, the buffer solution is phosphate-buffered saline.

In some embodiments, the detector molecules coupled to the first subset of molecular complexes are each coupled to a binding molecule of the associated molecular complex. For example, in some embodiments, coupling the detector molecules to the first subset of molecular complexes includes coupling the detector molecules to one or more binding molecules thereof. For example, as shown and described above with reference to FIGS. 1 and 3A-5B, the detector molecules 130 each couple to one or more binding molecules 125.

In some embodiments, the method 600 includes coupling the molecular complex to the sensor surface before coupling one or more detector molecules to the first subset of molecular complexes. Alternatively, the method 600 may include coupling the molecular complex to the sensor surface after coupling one or more detector molecules to the first subset thereof.

In some embodiments, coupling the detector molecules to the first subset of molecular complexes includes generating one or more modified detector molecules by adding a tag thereto and coupling the tagged detector molecules to a binding molecule (e.g., via the tag), of the molecular complex. Alternatively, the tagged detector molecules can be bound directly to the sensor surface.

In some embodiments, coupling the detector molecules to the first subset of molecular complexes occurs in the presence of an electric field. Alternatively, the electric field can be used to bind the detector molecules directly to the sensor surface.

The method 600 further includes coupling (step 660) one or more passivation agents to a second subset of the molecular complexes, which may be distinct from or overlap the first subset of the molecular complexes. In some embodiments, the passivation agent(s) include APA (PEG5), amino-PEG5-alcohol, or other species that keeps molecules other than the target away from the sensor surface.

In some embodiments, coupling the one or more passivation agents to the second subset of the molecular complexes includes generating a passivation agent mixture by dissolving, at least in part, the one or more passivation agents in a buffer solution, applying the mixture to the second subset of molecular complexes, and incubating for a second predetermined amount of time at a predetermined temperature. In some embodiments, the second predetermined amount of time is at least 15 minutes, and the predetermined temperature is 4° C. In some embodiments, the passivation agent mixture has a concentration of 3% and a pH balance 8.

In some embodiments, the passivation agents are coupled to a binding molecule of the associated molecular complex. For example, coupling the passivation agent(s) to the second subset of molecular complexes may include coupling to one or more binding molecules of the second subset of molecular complexes. As shown and described above with reference to FIGS. 1 and 3A-5B, the passivation agents 135 may couple to one or more binding molecules 125. Alternatively, the passivation agents 135 may couple to one or more linker molecules 120.

In some embodiments, the method 600 includes coupling the molecular complexes to the sensor surface before coupling one or more passivation agents to the second subset thereof. In some embodiments, the method 600 includes coupling the molecular complexes to the sensor surface after coupling one or more passivation agents to the second subset thereof.

The first and second subsets of molecular complexes may be present in a predetermined ratio. For example, in some embodiments, the population of the first subset of molecular complexes can be less than, equal to, or greater than the population of the second subset of molecular complexes. In some embodiments, the predetermined ratio is 60% of the first subset of molecular complexes to 40% of the second subset of molecular complexes, 80% of the first subset of molecular complexes to 20% of the second subset of molecular complexes, 100% of the first subset of molecular complexes to 0% of the second subset of molecular complexes, 40% of the first subset of molecular complexes to 60% of the second subset of molecular complexes, etc.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

What is claimed is:

1. A method of functionalizing a graphene sensor surface, the method comprising:
providing a graphene layer including a surface;
binding a plurality of molecular complexes to the surface of the graphene layer, wherein each molecular complex includes:
a linker molecule configured to couple to the sensor surface at a first linker position, and
a binding molecule coupled to the linker molecule at a second linker position different from the first linker position;
coupling one or more detector molecules to a first subset of the molecular complexes; and
coupling one or more passivation agents to a second subset of the molecular complexes, at least some of the molecular complexes of first subset being different from molecular complexes of the second subset.

2. The method of claim 1, wherein the one or more detector molecules coupled to the first subset of the molecular complexes are each coupled to a binding molecule of the associated molecular complex.

3. The method of claim 1, wherein the one or more passivation agents coupled to the second subset of the molecular complexes are each coupled to a binding molecule of the associated molecular complex.

4. The method of claim 1, further comprising coupling the molecular complex to the sensor surface before coupling one or more detector molecules to the first subset of the molecular complexes.

5. The method of claim 1, further comprising coupling the molecular complex to the sensor surface after coupling one or more detector molecules to the first subset of the molecular complexes.

6. The method of claim 1, further comprising coupling the molecular complex to the sensor surface before coupling one or more passivation agents to the second subset of the molecular complexes.

7. The method of claim 1, further comprising coupling the molecular complex to the sensor surface after coupling one or more passivation agents to the second subset of the molecular complexes.

8. The method of claim 1, wherein binding the plurality of molecular complexes to the sensor surface of the graphene layer includes:
generating a molecular complex mixture by dissolving, at least in part, the plurality of molecular complexes in a buffer solution;
disposing the molecular complex mixture on the sensor surface of the graphene layer; and
incubating the molecular complex mixture and the graphene layer for a first predetermined amount of time at a predetermined temperature.

9. The method of claim 8, wherein the first predetermined amount of time is at least 14 hours and the predetermined temperature is 4° C.

10. The method of claim 8, wherein the molecular complex mixture has a molar concentration of approximately 5 millimolar.

11. The method of claim 1, wherein coupling the one or more detector molecules to the first subset of the plurality of molecular complexes includes:
generating a detector molecule mixture by dissolving, at least in part, the one or more detector molecules in a buffer solution;
disposing the detector molecule mixture on the first subset of the molecular complexes; and
incubating the detector molecule mixture and the first subset of the molecular complexes for a second predetermined amount of time at a predetermined temperature.

12. The method of claim 11, wherein detector molecule mixture has a predetermined mass concentration of 10 μg/ml.

13. The method of claim 1, wherein coupling the one or more passivation agents to the second subset of the molecular complexes includes:
generating a passivation agent mixture by dissolving, at least in part, the one or more passivation agents in a buffer solution;
disposing the passivation agent mixture on the second subset of the molecular complexes; and
incubating the passivation agent mixture and the second subset of the molecular complexes for a second predetermined amount of time at a predetermined temperature.

14. The method of claim 13, wherein the passivation agent mixture has a predetermined concentration of 3% and a pH balance of 8.

15. The method of claim 1, wherein coupling the one or more detector molecules to the first subset of the plurality of molecular complexes includes:
generating one or more modified detector molecules by adding a respective tag to the one or more detector molecules; and
coupling the one or more modified detector molecules to one or more binding molecules of the first subset of the plurality of molecular complexes via respective tags of the one or more modified detector molecules.

16. The method of claim 1, wherein coupling the one or more detector molecules to the first subset of the molecular complexes includes, while generating an electric field, coupling the one or more detector molecules to one or more binding molecules of the first subset of the molecular complexes.

17. The method of claim 1, the first subset of the plurality of molecular complexes and the second subset of the molecular complexes have a predetermined ratio.

18. The method of any of claims 1, wherein the plurality of molecular complexes are configured to control an orientation of the one or more detector molecules.

19. The method of claim 1, wherein the one or more detector molecules include one of an antibody, enzyme, or protein.

20. The method of claim 1, wherein the binding molecule includes one of protein A, protein G, or protein L.
